(19) United States
(12) Patent Application Publication
(10) Pub. No.: US 2008/0004436 A1
(43) Pub. Date: Jan. 3, 2008

(54) Directed Evolution and Selection Using in Vitro Compartmentalization

(75) Inventors: Dan Tawfik, Jerusalem (IL); Kalia Bernath, Mazkeret-Batia (IL); Shlomo Magdassi, Jerusalem (IL); Sergio Gerardo Peisajovich, Rehovot (IL)

Correspondence Address: FENNEMORE CRAIG, 3003 NORTH CENTRAL AVENUE, SUITE 2600, PHOENIX, AZ, 85012, US

(73) Assignees: YEDA RESEARCH AND DEVELOPMENT CO. LTD. at the Weizmann Institute of Science, Rehovot (IL); YISSUM RESEARCH DEVELOPMENT COMPANY OF THE HEBREW UNIVERSITY OF JERUSALEM, Jerusalem (IL)

(21) Appl. No.: 11/718,929

(22) PCT Filed: Nov. 15, 2005
(86) PCT No.: PCT/IL05/01207

§ 371 Date: Aug. 7, 2007

Related U.S. Application Data
(60) Provisional application No. 60/627,185, filed on Nov. 15, 2004.

(51) Int. Cl. C07H 21/00

(52) U.S. Cl. 536/23.1

(57) ABSTRACT

The present invention is related to the field of compartmentalized libraries of genetic elements and the selection of biologically active molecules and the genes encoding same from said libraries. The selection assay of the invention utilizes water-in-oil emulsions and is particularly advantageous in applications in the field of directed-evolution, as exemplified herein for selection of protein inhibitors of DNA nucleases.

### FIELD OF THE INVENTION

The present invention is related to the field of compartmentalized libraries of genetic elements and the selection of biologically active molecules and the genes encoding same from said libraries. The selection assay of the invention utilizes water-in-oil emulsions and is particularly advantageous in applications in the field of directed-evolution, as exemplified herein for selection of protein inhibitors of DNA nucleases.

### BACKGROUND OF THE INVENTION

There exist a number of high-throughput display selection strategies based on a physical linkage between the gene and the protein it encodes (Griffiths, A. D. and Tawfik, D. S. (2000). Curr Opin Biotechnol 11, 338-53). These provide a powerful means of selecting proteins that bind any given ligand. However, the established rule of ‘you get what you select for’ surmises that indirect selections are generally ineffective. Thus, selections of enzymatic activities merely via assessment of binding abilities (e.g., to substrates or inhibitors) are less effective than a direct selection for high turnover rates (Griffith and Tawfik, 2000, op. cit.). Similarly, a selection for inhibitors by binding to the target enzyme may yield proteins or peptides that although they tightly bind the enzyme, are poor inhibitors since they bind outside the relevant/active-site.

A system based on in vitro compartmentalization (IVC) developed by one of the inventors of the present invention, is disclosed in Tawfik et al. (Nat Biotechnol 16, 652-6, 1998). The IVC system provides a flexible mean of linking genotype to phenotype which enables selection not only according to binding (as with other in vitro approaches) but also in accordance with enzymatic regulatory and inhibitory activities as demonstrated in Ghadessy et al. (Proc Natl Acad Sci USA 98, 4552-7, 2001); Sepp, et al. (FEBS Lett 532, 455-8, 2002); Lee et al. (Nucleic Acids Res 30, 4937-44, 2002); Griffiths and Tawfik (Embo J 22, 24-35, 2003); Yanagawa et al. (Nucleic Acids Res 31, e118, 2003 and Nucleic Acids Res 32, e95, 2004); and Cohen et al. (Protein Engineering Design & Selection 17, 3-11, 2004). The basic concept is simple: water-in-oil (w/o) emulsions of more than 10¹⁰ aqueous micro-droplets in 1 milliliter of oil are formed. In these artificial cell-like micro-droplets (compartments), approximately 2 μm in diameter and having a volume of about 5 femtoliter, a variety of biochemical processes take place while the external oil phase remains inert. IVC was therefore applied to select binding as well as enzymatic activities.

Water-in-oil emulsions for compartmentalization and for selection of genes having a pre-determined function from large gene libraries are known in the art, as disclosed for example in U.S. Pat. Nos. 6,495,673; 6,489,103; 6,184,012; 5,766,861 and US Patent Application No. 2003/0124586 to one of the inventors of the present invention and others. The aqueous droplets of the water-in-oil emulsion function as cell-like compartments in which a single gene being transcribed and translated to give multiple copies of the gene product (e.g., an enzyme). The contents of U.S. Pat. No. 6,495,673; U.S. Pat. No. 6,489,103; U.S. Pat. No. 6,184,012; U.S. Pat. No. 5,766,861 and US 2003/0124586 are incorporated herein by reference as if fully set forth in their entirety.

WO 2005/049787 of the inventors of the present invention and others discloses an in vitro system based on a library of molecules or cells, the library includes a plurality of distinct molecules or cells encapsulated within a water-in-oil-in-water emulsion. The emulsion includes a continuous external aqueous phase and a discontinuous dispersion of water-in-oil droplets. The internal aqueous phase of a plurality of such droplets comprises a specific molecule or cell from the library. WO 2005/049787 is incorporated herein in its entirety by reference.

In vitro compartmentalization (IVC) as disclosed in WO99/02671 to one of the inventors of the present invention, uses water-in-oil emulsions to create artificial cell-like compartments in which genes can be individually transcribed and translated. However, the genes in this IVC system must be linked (i.e. within the same compartment) to their gene products for the purpose of selection and detection.

Whilst compartmentalization ensures that the gene, the protein it encodes and the products of the activity of this protein remain linked, it does not afford a way of selecting based on the desired activity itself. Thus, there is an unmet need for compartmentalization systems enabling selection of a gene product for a desired activity, from a library of genes.

### SUMMARY OF THE INVENTION

The present invention provides an in vitro system for compartmentalization of large molecular libraries and provides methods for selection and isolation of molecules having desired activities from such libraries.

The present invention provides novel and inventive applications of IVC for the selection of molecules being capable of modulating a particular activity of a known biologically active moiety, including, but not limited to an enzyme. The inventors of the present invention utilize a micelle delivery system that enables the transport of various solutes, including metal ions, into the emulsion droplets thereby inducing a desired activity of the known biologically active moiety or of the gene product. Surprisingly, using this transport mechanism enables activation of the biologically active moiety selection of gene products by their activity.

The present invention is based ion part on the unexpected finding that an IVC system can be used for directed evolution of nuclease inhibitors. The inventors utilized an IVC system consisting of a water-in-oil emulsion comprising aqueous droplets having the following components: (1) genetic elements from a gene library encoding nuclease inhibitors and variants thereof; (2) the components required for in vitro transcription and translation; and (3) inactive nucleases. The system was incubated under conditions enabling transcription and translation of the genetic elements within the aqueous droplets. The inactive nucleases were then activated by merging micelles comprising bivalent metal ions (e.g. nickel or cobalt) into the aqueous droplets. Following digestion of genetic elements by the activated nuclease, only genes that survived the digestion, i.e. genes encoding nuclease inhibitors, were amplified, detected and isolated. This assay selection was directed explicitly for the desired activity, i.e. nuclease inhibition, and not merely for binding between a gene product and the nuclease. The stringency of selection can be easily modulated to give high enrichments (100-500 fold) and recoveries.

The delivery system of the present invention may contain any desired solute and may be merged with any emulsion for the purpose of introducing the solute to the internal discontinuous aqueous phase of an emulsion. Similarly, the method of the invention may be used for selecting any moiety according to the biological activity thereof, following the principles of the invention.

It is to be understood that colicin, colicin variants and libraries of the gene encoding the cognate inhibitor of colicin E9 (immunity protein 9, or Im9) for inhibition of another colicin (ColE7), merely serve to demonstrate the delivery system of the invention and the utility thereof for selection of molecules having a desired activity.

According to one aspect, the present invention provides a library of genetic elements encoding gene products, the library being compartmentalized in aqueous droplets of a water-in-oil emulsion, wherein each aqueous droplet comprises the components necessary to express gene products encoded by the genetic elements and further comprises at least one biologically active moiety the activity of which results in the modification of said genetic elements or the gene products encoded by said genetic elements.

According to one embodiment, the at least one biologically active moiety is not active. According to yet another embodiment, each aqueous droplet further comprises at least one activating agent capable of activating the biologically active moiety. According to yet another embodiment, the at least one biologically active moiety is selected from the group consisting of: a protein, a polypeptide and a peptide. According to yet another embodiment, the at least one biologically active moiety is an enzyme. According to yet another embodiment, the at least one biologically active moiety is a nuclease.

According to yet another embodiment, the at least one activating agent is selected from the group consisting of: inorganic or organic salts, monosaccharides, disaccharides, oligosaccharides, amino acids, peptides, polypeptides, nucleotides, nucleosides, oligonucleotides, polynucleotides, vitamins, and small organic molecules. According to yet another embodiment, the at least one biologically active moiety is a nuclease and the at least one activating agent is a bivalent salt.

According to another aspect, the present invention provides a method for selecting genetic elements encoding gene products of a desired activity, the method comprising:
a) providing a library of genetic elements;
b) providing at least one biologically active moiety the activity of which results in the modification of said genetic elements or the gene products encoded by said genetic elements;
c) co-compartmentalizing the genetic elements with the at least one biologically active moiety into droplets, the aqueous droplets being the internal discontinuous phase of a water-in-oil emulsion, such that each aqueous droplet comprises at least one genetic element together with the at least one biologically active moiety and further comprises components necessary to express the gene products encoded by said at least one genetic element;
d) merging the water-in-oil emulsion with micelles comprising at least one activating agent capable of modulating the activity of said at least one biological moiety; and
e) detecting genetic elements encoding gene products having a desired activity.

According to one embodiment the method further comprises, prior to merging the water-in-oil emulsion with the micelles, the step of
incubating the water-in-oil emulsion under conditions enabling expression of said gene products.

According to another embodiment the method further comprises, following merging the water-in-oil emulsion with the micelles, the steps of:
coalescing the water-in-oil emulsion thereby forming a continuous aqueous phase from the droplets; and
detecting in the aqueous phase genetic elements which encode the desired gene products.

According to yet another embodiment, detecting the genetic elements is performed by amplifying said genetic elements using PCR techniques and detecting the amplified products.

According to an alternative embodiment, the aqueous phase is re-emulsified prior to amplification. According to one embodiment, the aqueous phase is re-emulsified in oil comprising a surfactant capable of maintaining the integrity of the water-in-oil emulsion at temperatures within the range of 65° C. to 100° C. According to yet another embodiment, the surfactant is a polymer having a Hydrophilic-Lipophilic Balance (HLB) value below 10. According to certain embodiments, the HLB value is within the range of 3 to 6. According to yet another embodiment, the surfactant is high molecular weight modified polyether polysiloxane. According to yet another embodiment, the surfactant is selected from the group consisting of: cetyl dimethicone copolyol, polysiloxane polyalkyl polyether copolymer, cetyl dimethicone copolyol, polyglycerol ester, poloxamer and polyvinyl pyrrolidone (PVP)/hexadecane copolymer. According to yet another embodiment, the surfactant is cetyl dimethicone copolyol. According to yet another embodiment, the content of said surfactant in the oil is within the ranges of 1-20% v/v.

According to yet another embodiment, detecting said genetic elements is carried out by a technique selected from: plasmid nicking assay and capture of surviving genes on magnetic beads following amplification by PCR.

According to yet another embodiment, the micelles comprise from 100 to 400 volumes of oil, and from 10 to 40 volumes of total surfactant to every one volume of an aqueous phase containing the at least one activating agent. According to another embodiment, the micelles have a mean droplet size in the range of 0.01 micron to 1 micron. According to a particular embodiment, the mean droplet size is approximately 0.1 micron.

According to yet another aspect the present invention provides a product selected according to the method of the invention. As used in this context, a “product” may refer to a gene product selectable according to the method of the invention or the genetic element (or genetic information comprised therein.) According to certain embodiments, the product in a nuclease inhibitor.

### BRIEF DESCRIPTION OF THE DRAWINGS

FIG. 1 is a schematic view of the selection system wherein a library of genes is added to a cell-free translation extract, and compartmentalized in the aqueous droplets of a water-in-oil (w/o) emulsion together with an inactive DNase and after the genes are allowed to transcribe and translate, the DNase is activated through the delivery of nickel or cobalt ions by micelles (micelles) and genes encoding a DNase inhibitor survive the digestion and are subsequently isolated and amplified by PCR.

FIG. 2 presents size distribution of the nickel ion micelles.

FIG. 3 exhibits model selections for the gene encoding the inhibitor Im9 wherein A is gel analysis of the PCR-amplified DNA (M, Marker DNA (100 bp GeneRuler™, Fermentas); ‘Unselected’ refers to a sample containing Im9 and ΔOPD biotinylated genes at a ratio of 1:200, emulsified without ColE9 extract; ‘DNA mix’ refers to the original mixture of genes amplified with no prior treatment) and B is the level of survival of the gene in excess (ΔOPD) as determined by competitive PCR.

FIG. 4 demonstrates selectivity and stringency of the selection pressure.

FIG. 5 presents the progress of the selection of Im9 libraries for inhibition of ColE7.

FIG. 6 exhibits the diminishing of inhibition activity of the evolved variant #8 in presence of ColE9H127A mutant.

FIG. 7 demonstrates selection for higher selectivity.

FIG. 8 shows the stability of cetyl dimethicone copolyol-based emulsions after 32 PCR cycles: (A) droplet size, determined by Dynamic Light Scattering, before (solid line) and after (dashed line) 32 PCR cycles; (B) appearance of the emulsion under the microscope, before (left) and after (right) 32 PCR cycles.

FIG. 9 presents the stability of cetyl dimethicone copolyol-based emulsions in two separate emulsions (A), the first emulsion containing a long template with all the components necessary for amplification and the second emulsion containing a shorter template and is devoid of the primers required for amplification and the PCR products obtained from these emulsions (B) or from positive control emulsions (C).

FIG. 10 is a schematic representation of two DNA templates being used for demonstrating the ability of cetyl dimethicone copolyol-based emulsions to prevent recombination artifacts (A), the expected sizes of the PCR products (B) and the two intermediate-size bands arising from recombination artifacts of the two original templates (C), as follows: amplification product of the emulsion containing the “long DNA template 2”, lane 1; amplification product of the emulsion containing the “short DNA template 2”, lane 2; amplification products of the emulsion containing both DNA templates, lane 3; amplification product of a non-emulsified mixture containing both DNA templates, lane 4.

### DETAILED DESCRIPTION OF PREFERRED EMBODIMENTS

Definitions

The term “emulsion” as used herein is in accordance with the meaning normally assigned thereto in the art and further described herein. In essence, however, an emulsion may be produced from any suitable stable combination of immiscible liquids. Typically, the emulsion of the present invention has an aqueous phase that contains the molecular components, as the dispersed phase present in the form of finely divided aqueous droplets (the disperse, internal or discontinuous phase), also termed hereinafter “microcapsules dispersed in oil” and further comprises a hydrophobic, liquid phase (an “oil”) as the matrix in which these droplets are suspended (the continuous or external phase). Such emulsions are termed herein “water-in-oil” (w/o). Advantageously, the entire aqueous phase containing the molecular components is compartmentalized in discrete droplets (the internal phase). The hydrophobic oil phase generally contains none of the biochemical components and hence is inert.

It is to be explicitly understood that emulsions may further comprise natural or synthetic emulsifiers, co-emulsifiers, stabilizers and other additives as are well known in the art.

The term “biologically active moiety” is used herein to describe a molecule, having an activity that results in the modulation of a gene or the products encoded by said gene, wherein upon such modulation the modulated (desired) gene or products can be distinguished from the non-modulated gene/products. Preferably, the biological active moiety is an enzyme capable of catalyzing changes in conformation, structure or amino acid content of the gene or the gene products. According to a preferred embodiment, the gene is a nuclease capable of catalyzing the degradation of the genetic elements. According to another preferred embodiment, the biologically active moiety is not part of the components required for in-vitro transcription and translation of the genetic elements within the aqueous droplets. According to yet another preferred embodiment, a non-active form of the biologically active moiety is co-compartmentalized with the genetic elements and is activated only after the genetic elements are allowed to transcribe and translate, thus enabling to select gene products that react with the biologically active moiety. Such gene products may be inhibitors, activators, inducers and/or regulators.

As used herein, a “genetic element” is a molecule, a molecular construct or a cell comprising a nucleic acid encoding a gene product. The genetic elements of the present invention may comprise any nucleic acid (for example, DNA, RNA or any analogue, natural or artificial, thereof). The nucleic acid component of the genetic element may moreover be linked, covalently or non-covalently, to one or more molecules or structures, including proteins, chemical entities and groups, solid-phase supports such as magnetic beads, and the like. In the methods of the invention, these structures or molecules can be designed to assist in the sorting and/or isolation of the genetic element encoding a gene product with the desired activity. It is further to be understood that the genetic elements of the present invention may be present within a cell, virus or phage.

The term “expression” as used herein, is used in its broadest meaning, to signify that a nucleic acid contained in the genetic element is converted into its gene product. Thus, where the nucleic acid is DNA, expression refers to the transcription of the DNA into RNA; where this RNA codes for protein, expression may also refer to the translation of the RNA into protein. Where the nucleic acid is RNA, expression may refer to the replication of this RNA into further RNA copies, the reverse transcription of the RNA into DNA and optionally the transcription of this DNA into further RNA molecule(s), as well as optionally the translation of any of the RNA species produced into protein. Preferably, therefore, expression is performed by one or more processes selected from the group consisting of: transcription, reverse transcription, replication and translation. Expression of the genetic element may thus be directed into DNA, RNA or protein, or a nucleic acid or protein containing unnatural bases or amino acids (the gene product) within the droplet of the invention, so that the gene product is confined within the same droplet as the genetic element. The genetic element and the gene product thereby encoded are linked by confining each genetic element and the respective gene product encoded by the genetic element within the same droplet. In this way the gene product in one droplet cannot cause a change in any other droplets.

A “library” refers to a collection of individual species distinct from one another in at least one detectable characteristic. The term “library” as used herein particularly refers to a gene library consisting of a plurality of distinct genetic elements. Other types of libraries are also encompassed within the scope of the present invention including libraries of viruses or phages and display libraries that include microbead-, phage-, plasmid-, or ribosome-display libraries and libraries made by CIS display and mRNA-peptide fusion. It is to be understood that that every member of the library does not have to be different from every other member. Often, there can be multiple identical copies of individual library members.

The term “variant” as used herein refers to a protein that possesses at least one modification compared to the original protein. Preferably, the variant is generated by modifying the nucleotide sequence encoding the original protein and then expressing the modified protein using methods known in the art. A modification may include at least one of the following: deletion of one or more nucleotides from the sequence of one polynucleotide compared to the sequence of a related polynucleotide, the addition of one or more nucleotides or the substitution of one nucleotide for another. Accordingly, the resulting modified protein may include at least one of the following modifications: one or more of the amino acid residues of the original protein are replaced by different amino acid residues, or are deleted, or one or more amino acid residues are added to the original protein. Other modifications may be also introduced, for example, a peptide bond modification, cyclization and circular permutation of the structure of the original protein. A variant may encompass all stereoisomers or enantiomers of the molecules of interest, either as mixtures or as individual species.

### DESCRIPTION OF THE PREFERRED EMBODIMENTS

The present invention provides a gene library of genetic elements encoding gene products, the library being compartmentalized in aqueous droplets of water-in-oil emulsions, wherein each aqueous droplet further comprises components necessary to express the gene products encoded by the genetic elements and further comprises at least one biologically active moiety capable of modulating the genetic elements or their gene products.

Water-in-oil emulsions as used herein for in vitro compartmentalization (IVC) are formed as disclosed in WO99/02671 with the exception, that the present invention does not require linkage between the genes and the corresponding transcribed and/or translated products. In principle, water-in-oil emulsions create artificial cell-like compartments in which genes can be individually transcribed and translated. Preferably, the emulsions are heterogeneous systems of two immiscible liquid phases with one of the phases dispersed in the other as droplets of microscopic or colloidal size.

Emulsions may be produced from any suitable combination of immiscible liquids. Preferably the emulsion of the present invention comprises water which encompass (a) the components required for in vitro transcription and translation; (b) the at least one biologically active moiety, the activity of which results in the modification of said genetic elements or the gene products encoded by said genetic elements; and (c) genetic elements from a gene library. In the emulsion, the water is the phase present in the form of finely divided droplets (the disperse, internal or discontinuous phase). The emulsion further comprises a hydrophobic, immiscible liquid (an ‘oil’) as the matrix in which these droplets are suspended (the nondisperse, continuous or external phase). Such emulsions are termed ‘water-in-oil’ (W/O). This has the advantage that the entire aqueous phase containing the (a) to (c) biochemical components listed above, is compartmentalized in discreet droplets (the internal phase). The external phase, being hydrophobic oil, generally contains none of the biochemical components and hence is inert.

The emulsion may be stabilized by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents. Suitable oils include light white mineral oil and non-ionic surfactants such as sorbitan monooleate (Span80; ICI) and polyoxyethylenesorbitan monooleate (Tween 80; ICI).

The use of anionic surfactants may also be beneficial. Suitable surfactants include sodium cholate and sodium taurocholate. Particularly preferred is sodium deoxycholate, preferably at a concentration of 0.5% w/v, or below. Inclusion of such surfactants can in some cases increase the expression of the genetic elements and/or the activity of the gene products.

Addition of some anionic surfactants to a non-emulsified reaction mixture completely abolishes translation. During emulsification, however, the surfactant is transferred from the aqueous phase into the interface and activity is restored. Addition of an anionic surfactant to the mixtures to be emulsified ensures that reactions proceed only after compartmentalization.

Creation of an emulsion generally requires the application of mechanical energy to force the phases together. There are a variety of ways of doing this, which utilize a variety of mechanical devices, including stirrers (such as magnetic stir-bars, propeller and turbine stirrers, paddle devices and whisks), homogenizers (including rotor-stator homogenizers, high-pressure valve homogenizers and jet homogenizers), colloid mills, and ultrasound and ‘membrane emulsification’ devices.

Aqueous microcapsules formed in water-in-oil emulsions are generally stable with little if any exchange of genetic elements or gene products between microcapsules. Additionally, it has been demonstrated that several biochemical reactions proceed in emulsion microcapsules.

Moreover, complicated biochemical processes, notably gene transcription and translation are also active in emulsion microcapsules. The technology exists to create emulsions with volumes all the way up to industrial scales of thousands of liters.

The preferred microcapsule size will vary depending upon the precise requirements of any individual selection process that is to be performed according to the present invention. In all cases, there will be an optimal balance between the size of the gene library, the required enrichment and the required concentration of components in the individual microcapsules to achieve efficient expression and reactivity of the gene products.

The processes of expression must occur within each individual microcapsule provided by the present invention. Both in vitro transcription and coupled transcription-translation become less efficient at sub-nanomolar DNA concentrations. Because of the requirement for only a limited number of DNA molecules to be present in each microcapsule, this therefore sets a practical upper limit on the possible microcapsule size. Preferably, the mean volume of the microcapsules is less that 5.2×10⁻¹⁶ m³, (corresponding to a spherical microcapsule of diameter less than 10 cm, more preferably less than 6.5×10⁻¹⁷ m³ (5 μm), more preferably about 4.2×10⁻¹⁸ m³ (2 m) and ideally about 9×10⁻¹⁸ m³ (2.6 μm).

The effective DNA or RNA concentration in the microcapsules may be artificially increased by various methods that will be well known to those versed in the art. These include, for example, the addition of volume excluding chemicals such as polyethylene glycols (PEG) and a variety of gene amplification techniques, including transcription using RNA polymerases including those from bacteria such as E. coli, eukaryotes and bacteriophage such as T7, T3 and SP6; the polymerase chain reaction (PCR) (Saiki et al., 1988); Qss replicase amplification; the ligase chain reaction (LCR); and self-sustained sequence replication system and strand displacement amplification. Even gene amplification techniques requiring thermal cycling such as PCR and LCR could be used if the emulsions and the in vitro transcription or coupled transcription-translation systems are thermostable (for example, the coupled transcription-translation systems could be made from a thermostable organism such as Thermus aquaticus).

Increasing the effective local nucleic acid concentration enables larger microcapsules to be used effectively. This allows a preferred practical upper limit to the microcapsule volume of about 5.2×10⁻¹⁶ m³ (corresponding to a sphere of diameter 10 μm).

The droplet size must be sufficiently large to accommodate all of the required components of the biochemical reactions that are needed to occur within the microcapsule. For example, in vitro, both transcription reactions and coupled transcription-translation reactions require a total nucleoside triphosphate concentration of about 2 mM.

For example, in order to transcribe a gene to a single short RNA molecule of 500 bases in length, this would require a minimum of 500 molecules of nucleoside triphosphate per droplet (8.33·10⁻²² moles). In order to constitute a 2 mM solution, this number of molecules must be contained within a droplet of volume 4.17·10⁻¹⁹ liters (4.17·10⁻²² m³ which if spherical would have a diameter of 93 nm).

Furthermore, particularly in the case of reactions involving translation, it is to be noted that the ribosomes necessary for the translation to occur are themselves approximately 20 nm in diameter. Hence, the preferred lower limit for primary droplets is a diameter of approximately 0.1 μm (100 nm). Therefore, the primary droplet volume is of the order of between 5.2·10⁻²² m³ and 5.2·10⁻¹⁶ m³ corresponding to a sphere of diameter between 0.1 μm and 10 μm, preferably of between about 5.2·10⁻¹⁹ m³ and 6.5·10⁻¹⁷ m³ (1 μm and 5 μm). Sphere diameters of about 2.6 μm are advantageous.

It is no coincidence that the preferred dimensions of the primary compartments (droplets of 2.6 μm mean diameter) closely resemble those of bacteria, for example, Escherichia are 1.1-1.5·2.0-6.0 μm rods and Azotobacter are 1.5-2.0 μm diameter ovoid cells. In its simplest form, Darwinian evolution is based on a ‘one genotype one phenotype’ mechanism. The concentration of a single compartmentalized gene, or genome, drops from 0.4 nM in a compartment of 2 μm diameter, to 25 μM in a compartment of 5 μm diameter. The prokaryotic transcription/translation machinery has evolved to operate in compartments of about 1-2 μm diameter, where single genes are at approximately nanomolar concentrations. A single gene, in a compartment of 2.6 μm diameter is at a concentration of 0.2 nM. This gene concentration is high enough for efficient translation. Compartmentalization in such a volume also ensures that even if only a single molecule of the gene product is formed it is present at about 0.2 nM, which is important if the gene product is to have a modifying activity of the genetic element itself. The volume of the primary droplet should thus be selected bearing in mind not only the requirements for transcription and translation of the genetic element, but also the modifying activity required of the gene product in the method of the invention.

The size of emulsion microcapsules may be varied simply by tailoring the emulsion conditions used to form the emulsion according to requirements of the selection system. The larger the microcapsule (i.e. aqueous droplet) size, the larger is the volume that will be required to encapsulate a given genetic element library, since the ultimately limiting factor will be the size of the microcapsule and thus the number of microcapsules possible per unit volume.

The size of the aqueous droplets is selected not only having regard to the requirements of the transcription/translation system, but also those of the selection system employed for the genetic element. Thus, the components of the selection system, such as a chemical modification system, may require reaction volumes and/or reagent concentrations that are not optimal for transcription/translation. As set forth herein, such requirements may be accommodated by a secondary re-encapsulation step; moreover, they may be accommodated by selecting the microcapsule size in order to maximize transcription/translation and selection as a whole. Components necessary to express the gene products encoded by the at least one genetic element in each aqueous droplet of the water in oil emulsion will for example comprise those necessary for transcription and/or translation of the genetic element. These are selected from the following: a suitable buffer, an in vitro transcription/replication system and/or an in vitro translation system containing all the necessary ingredients, enzymes and cofactors, RNA polymerase, nucleotides, nucleic acids (natural or synthetic), transfer RNAs, ribosomes and amino acids, and the substrates of the reaction of interest in order to allow selection of the modified gene product.

A suitable buffer will be one in which all of the desired components of the biological system are active and will therefore depend upon the requirements of each specific reaction system. Buffers suitable for biological and/or chemical reactions are known in the art and recipes provided in various laboratory texts.

The in vitro translation system will usually comprise a cell extract, typically from bacteria (Zubay, Annu Rev Genet., 7:267-287, 1973; Lesley et al., J Biol. Chem., 266(4):2632-2638), rabbit reticulocytes (Pelham and Jackson, Eur J. Biochem., 67(1):247-256, 1976), or wheat germ. Many suitable systems are commercially available (for example from Promega) including some which will allow coupled transcription/translation (all the bacterial systems and the reticulocyte and wheat germ TNT™ extract systems from Promega). The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected (Ellman et al., Methods Enzymol., 202:301-336, 1991; Mendel et al., Annu Rev Biophys Biomol Struct., 24:435-462, 1995).

Preferably, the biologically active moiety is inactive, and its activity is modulated upon merging the compartmentalized library with a solution of micelles (also termed herein “micelles”) comprising one or more activating agent. The micelles typically have a mean droplet size in the submicron range. The compartmentalized library of the present invention provides a general means of regulating biochemical processes that occur within the cell-like compartments and is of much utility.

The present invention further provides a new use of IVC the principles of which are exemplified in the direct selection of nuclease inhibitors: a library of genes was compartmentalized, single genes were allowed to transcribe and translate within aqueous droplets that also contain a non-active DNA-nuclease such that, genes encoding a peptide or protein that inhibits the nuclease survived, whilst other genes, that do not encode an inhibitor, were digested. This strategy requires a regulatory mechanism that activates the nuclease only after gene translation has been completed. Or else, all genes would be indiscriminately digested before they had the chance to be translated. The delivery system of the present invention overcomes this deficiency as it is based on the solubilization of water-soluble ions in micelles (or swollen micelles) and the merging of these droplets with the aqueous droplets of the IVC emulsion, thus enabling the user monitoring processes within the emulsion droplets after their formation.

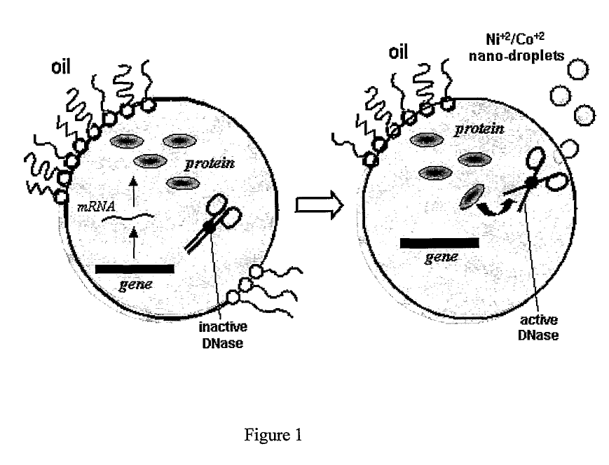

The advantages and utility of the system of the present invention is demonstrated in a system for the selection of inhibitors for colicin DNases (ColEs) utilizing bivalent metal ions such as nickel or cobalt, that can be delivered by micelles, for activating ColEs. The selection method of the invention is schematically presented in FIG. 1. Briefly, using these particular molecules, the in-vitro evolved inhibitors showed significant inhibition of ColE7 both in vitro and in vivo. These Im9 variants carry mutations into residues that determine the selectivity of the natural counterpart (Im7) while completely retaining the residues that are conserved throughout the family of immunity protein inhibitors. The in vitro evolution process confirms earlier hypotheses regarding the ‘dual recognition’ binding mechanism and the way by which new colicin-immunity pairs diverged from existing ones.

It is noted that although the principles of the invention are exemplified herein below for colicin endonucleases and their natural inhibitors for illustrative purposes only and should not be construed in a limitative fashion.

The colicin endonucleases and their natural inhibitors, namely, the immunity proteins that were explored, were chosen for the purpose of demonstration as they comprise an interesting system of molecular synergism evolved by nature. Colicin endonucleases are used by E. coli to kill competing bacterial strains under stress conditions. The immunity proteins (Im) provide protection to the attacking bacteria from destruction of their own DNA. Following the co-expression and secretion of the ColE-Im complex, the ColE is released from its Im inhibitor, and is free to attack other bacteria. There are 4 known pairs of DNase ColE-Im in E. coli, although many more pairs probably exist in nature. These cognate pairs bind with extremely high affinity (Kₐ≧10¹⁴ M⁻¹) and selectivity (binding of non-cognate partners is 10⁶-10¹⁰ fold weaker than cognate binding).

The in vitro selection system described here exhibits high enrichments and a wide dynamic range as demonstrated in model selections of genes encoding a cognate vs. a non-cognate immunity. Selection for the inhibitor is direct—genes are selected by virtue of their ability to encode a protein that inhibits the DNA nuclease activity, rather than simply bind the ColE. This system was applied to reproduce the process of evolution of one immunity protein into another. Specifically, Im9 (the cognate inhibitor of ColE9) was evolved towards inhibition of ColE7. The inventors of the present invention found that the newly evolved Im proteins accumulated mutations primarily in the ‘variable region’—a domain of immunity proteins that is thought to mediate specific, cognate binding. In contrast, no significant changes were observed in residues of the ‘hot spot’ that is highly conserved amongst all immunity proteins and mediates cross-reactivity between non-cognate pairs. These results provide strong support to the hypothesis of ‘dual recognition’ whereby the ‘conserved hot spot’ serves as a common anchoring point between all ColEs and Im proteins, and the ‘variable region’ provides the basis for selective recognition between cognate pairs, and mediates the divergent evolution of new ColE-Im pairs.

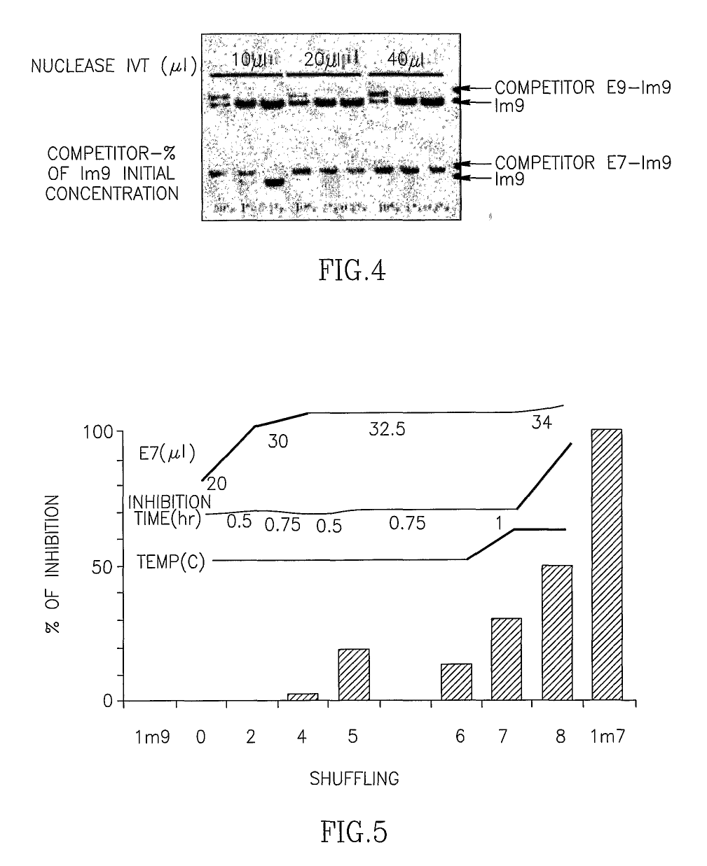

Previous selections for nuclease inhibitors, including Im proteins, were performed using phage-display libraries and a selection for binding of the nuclease. In contrast, the micelle delivery system of the present invention enables establishing a direct in vitro selection for the inhibition of DNA nucleases, as exemplified hereinbelow. This selection system affords good enrichment factors (100-500 fold) and good recovery of inhibitor-encoding genes (˜20%). The enrichment factor could be easily regulated in model selections of wild-type immunity genes (FIG. 4), as well as in library selections for new immunity protein variants (FIG. 5). In particular, adding higher volumes of ColE cell-free extracts does not only increase the number of ColE molecules per compartment, but also reduces the translation efficiency and hence the number of Im protein molecules. This results in a significant decrease in the Im/ColE ratio and thereby increases the stringency of selection and enrichment for high-affinity variants. This selection strategy may be applicable to other DNA-nucleases (be it endo- or exo-nuclease) and perhaps to other DNA-modifying enzymes (DNA-methyltransferases, for example).

The compartmentalized library of the invention and the selection method using same are advantageous over other systems and methods known in the art for at least the following reasons:
1. The compartmentalized library and the selection method of the invention enable screening for a specific function which may be mediated by more than one member of the library, rather than screening merely for binding.
2. The compartmentalized library and the selection method of the invention enable selection of a genetic element encoding a desired gene product without the need to label the desired product or gene encoding same. Moreover, detection of the desired moiety does not require induction of a detectable property such as an optical property of the moiety. This advantage is exemplified herein by the selection of specific nuclease inhibitors.
3. Use of the compartmentalized library and the selection method of the invention are particularly advantageous for selection of functional moieties that are fatal or essential to living cells. Selection of such moieties may be carried out only in vitro and moreover only in assays and systems that enable selection by function, as the teaching of the present invention.
4. Applying the selection method of the invention, using the cetyl dimethicone copolyol for re-emulsification prior to amplification by PCR overcomes the deficiencies of other emulsions known in the art, since the cetyl dimethicone copolyol emulsion remains stable even under PCR cycles, particularly during the high temperature required for DNA denaturation (about 94° C.).
5. Using the cetyl dimethicone copolyol for re-emulsification prior to amplification by PCR also provides an improved isolation of individual DNA molecules within the boundaries of the aqueous droplets, therefore significantly reduces recombination artifacts that may be introduced during PCR.

The compartmentalized library of the present invention enables activating the compartmentalized moiety while not affecting the integrity of the compartments. Previous works indicated few other ways of modulating the emulsion content without affecting its integrity. These include the delivery of hydrophobic substrates through the oil phase, reduction of pH by delivery of acetic acid, and photoactivation of a substrate contained within in the aqueous droplets (Griffiths and Tawfik, 2003, op. cit.).

The micelle (micelles) delivery used in the methods of the present invention significantly expands the scope of regulatory mechanisms. The high enrichment factors and recoveries indicate that the addition of micelles of the type described above to water-in-oil emulsions has no undesirable effects on the integrity of the aqueous compartment or exchange of genes and proteins between droplets. The delivery of a variety of low-molecular-weight, water-soluble ligands may also be helpful in regulating enzyme activities (by delivering allosteric effectors, for example) or gene expression (e.g., by IPTG-induced transcription of genes in cell-free extracts). Moreover, micelles as carriers into multiple emulsions were already reported for a variety of water soluble reagents as well as enzymes. Various compositions of micelles or swollen micelles allow high-molecular-weight molecules, e.g., DNA and proteins, to be delivered, as already shown for entrapment of glucose oxidase. The delivery of proteins or genes into emulsion droplets would be of much utility provided that it does not mediate the exchange of DNA or proteins between droplets and the subsequent loss of genotype-phenotype linkage.

Typically, the micelles which encompass the at least one activating agent comprise from 100 to 400 volumes of oil, and from 10 to 40 volumes of total surfactant to every one volume of an aqueous phase containing the solutes. According to some embodiments, the micelles have a mean droplet size in the range of 0.01 micron to 1 micron. According to a particular embodiment, the mean droplet size is approximately 0.1 micron. The activating agent within the micelles is selected from the group consisting of: inorganic or organic salts, monosaccharides, disaccharides, oligosaccharides, amino acids, peptides, polypeptides, nucleotides, nucleosides, oligonucleotides, polynucleotides, vitamins and small organic molecules. According to yet another embodiment, the solutes within the micelles are bivalent salts. As such, the solute may exhibit a variety of activities and thus may act as any one of the following: transmitors, activators, inducers and/or regulators of biological processes such as transcription among other enzymatic activities.

The selection method of the present invention is based on the amplification of the genes that survive ColE digestion by the Polymerase Chain Reaction (PCR). Amplification of the desired genetic elements resulting from the selection method of the invention may be carried out directly subjecting the aqueous solution obtained from coalescence of the aqueous droplets to PCR.

PCR has revolutionized biology, dramatically expanding our abilities to detect specific DNA molecules present in complex mixtures and manipulate them to our wish. However, as any other technique dealing with biological complexity, PCR is not free of problems. In particular, co-amplification of several closely-related templates with universal primers is known to generate recombination artifacts, due to: (i) premature termination during chain elongation, resulting in an incompletely extended product that acts as primer on a heterologous template; and (ii) cross-hybridization of heterologous sequences, leading to heteroduplex formation. The latter could become a single chimeric sequence following cloning, transformation and excision repair within a bacterial host. Recombination artifacts could lead to the wrong identification of unreal genetic diversity, particularly when analyzing: (i) genetic variation within cell populations, (ii) splice variants in heterogeneous tissues, and (iii) re-arrangement of immunoglobulin genes, among others. Different strategies have been devised to circumvent these problems; these include the engineering of improved polymerases with enhanced procesivities, the minimization of the number of cycles during the PCR reaction, or the development of specialized amplification protocols, such as “reconditioning PCR”. However, as long as multiple heterologous templates are still present within the amplification mixture, none of these methods can completely ensure the elimination of recombination artifacts.

A more promising strategy is based on the amplification of single molecule DNA templates within the aqueous compartments of a water-in-oil emulsion (emulsion PCR, or ePCR). As each individual DNA molecule is amplified within the boundaries of an aqueous droplet; the possibility of recombination artifacts should be drastically reduced. This method, as currently used, has been inspired by the development of in vitro compartmentalization for the transcription and translation of individual genes (Ghadessy 2001, ibid), and had found a variety of applications including in the identification of rare cancerous cells amongst large populations of normal cells, and in novel, high-throughput DNA sequencing strategies.

The use of ePCR can also prove beneficial in the amplification of genes selected in vitro, in compartmentalized, or any other in vitro system. This is particularly so, in those cases where genes carrying beneficial mutations (positives) are present at very low frequency, and the remaining population (negatives) carries a relatively high frequency of deleterious mutations (e.g., when libraries with high mutation load are selected). Since both the ‘positive’ and ‘negative’ genes are derived from the same gene, their co-amplification with the same primers, and in bulk solution, may result in recombination and in the loss of ‘positives’ due to the crossover with genes carrying deleterious mutation(s). As been observed by the inventors of the present invention, whilst a very small number (≧50) of ‘positive’ genes (e.g., genes encoding the DNA methyltransferase M.HaeIII) can be spiked into a large excess of a completely unrelated ‘negative’ gene (>10⁸), and subsequently recovered through 3-4 iterative rounds of selection, a similar, or even higher, number of wild type M.HaeIII genes cannot be recovered when spiked into an excess of ‘negative’ genes comprised of M.HaeIII genes carrying deleterious mutations.

Hence the inventors of the present invention surmised that, the application of ePCR for the amplification of library genes that are recovered from selection (and especially in the first rounds when ‘positive’ genes are still scarce) might be beneficial. However, the chemical composition of the emulsion used routinely for ePCR, a composition that is in fact rather similar to the one developed for selections at ambient temperatures, and is based on mineral oil and the surfactants Span 80, and Tween 80 or Triton X-100, is sub-optimal for PCR applications. Indeed, many conventional ethoxilated surfactants are very sensitive to high temperature, for instance Tween 80 that dehydrates at high temperatures, and thus are far from ideal for emulsions that should be stable at 94° C. The inadequacy of such components compromises the overall stability of the emulsion, and could lead to water droplet coalescence or to micellar exchange of water-phase components. An alternative formulation of an emulsion for performing PCR has been recently described, but the aqueous droplets obtained by this procedure are much larger (>>10 μm).

Surface-active agents (surfactants) are commonly added to the emulsion for stabilizing its compartmentalized structure. These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents. Particularly suitable oils include light white mineral oil and non-ionic surfactants such as sorbitan monooleate (Span™80; ICI) and polyoxyethylenesorbitan monooleate (Tween™ 80; ICI).

The present invention provides a novel emulsion formulation optimized for ePCR applications. The high stability of this formulation renders it ideal for the development of multiplex procedures for the isolation of single-cell DNA, RNA or protein, as well as for single-cell analysis at a population level.

As detailed above, current emulsions used for PCR are based on an oil phase composed of the surfactants Span80 (4.5% v/v), Tween 80 (0.5% v/v) and Triton X-100 (0.05% v/v), in mineral oil, a composition originally developed for in vitro transcription and translation applications and far from ideal for the high temperatures required for PCR-based applications. The stability during the PCR cycling of the emulsion used in the present invention is higher. The improvement is achieved by
(i) adding a polymeric surfactant with a longer hydrophobic tail, as this favors a higher separation between water droplets (steric stabilization), and
(ii) minimizing the presence of ethoxilated surfactants, such as Tween 80. Such surfactants became dehydrated in the high temperatures required for DNA denaturation in each PCR cycle, thereby destabilizing the emulsion.

Accordingly, the inventors of the present invention used mineral phases with different ratios of cetyl dimethicone copolyol (Abil® EM90) e.g. 1-3%, which is a high molecular weight modified polyether polysiloxane, (average MW˜1000), avoiding, at the same time, Tween 80. It is noted that Abil™ EM90 has been previously used for making emulsions and compartmentalizing in vitro translation reactions, in particular with eukaryotic cell-free translation systems such as the rabbit reticulocyte system (Ghadessy et al., Protein Engineering Design and Selection 17:201-204, 2004). However, the use of Abil® EM90 for emulsion PCR has not been described to date.

Other surfactant that may be used for the formation of emulsion suitable for ePCR are selected from the group consisting of: polysiloxane polyalkyl polyether copolymer, cetyl dimethicone copolyol, polyglycerol esters, poloxamers and PVP/hexadecane copolymers, such as Unimer U-151.

The nucleic acid portion of the genetic element may comprise suitable regulatory sequences, such as those required for efficient expression of the gene product, for example promoters, enhancers, translational initiation sequences, polyadenylation sequences, splice sites and the like.

### EXAMPLES

### Example 1

### The In Vitro Evolution of New Immunity Protein Variants

Initially, the ColE9 and Im9, ColE2 and ColE7 genes were PCR-amplified from plasmids pKC67, pKH202 and pColE2, respectively and cloned into pIVEX 2.2b (Roche) via NcoI and SacI sites to give pIVEX-E9, pIVEX-Im9, pIVEX-E2 and pIVEX-E7. Preparation of pIVEX-ΔOPD is described elsewhere (Griffiths and Tawfik, 2003, op. cit.). Im9 and ΔOPD PCR fragments for selection (FIG. 1) were amplified using primers LMB2-2 Bc appending a biotin (Biotin-5′-CAGGCTGCGCAACTGTTG-3′; SEQ ID NO:1) and LMB-3 (5′-GTCATAGCTGTTTCCTG-3′; SEQ ID NO:2). The reactions were cycled 30 times (95° C. 0.5 min, 55° C. 0.5 min, 72° C. for 0.5 min -2 min. depending on the fragments length). The ColE2, ColE7 and ColE9 genes were PCR-amplified from the ligation mixtures of pIVEX-E9, pIVEX-Im9, pIVEX-E2 and pIVEX-E7, using primers LMB2-6 (5′-ATGTGCTGCAAGGCGATT-3′; SEQ ID NO:3) and pIVB-6 (5′-GTCGATAGTGGCTCCAA-3′; SEQ ID NO:4).

DNA from error-prone libraries, and the surviving DNA from each round of selection, were virtually-cloned into pIVEX, and amplified with biotinylated primers as described above (Griffiths and Tawfik, 2003, op. cit.). The DIG-Biotin DNA substrate was amplified from a pIVEX vector carrying an insert which encodes the N-Flag and HA epitopes connected by a short linker, using primer LMB2-2 Bc appending a biotin, and LMB-3 appending a digoxegenin (DIG) at the 5′ end. The DNA fragments were all purified using the Wizard PCR Preps (Promega).

For the DNA digestion and nuclease activity assays, ColE, Im, and ΔOPD genes were translated separately in Promega's S30 Extract System for Linear Templates supplemented with T7 polymerase essentially as described (Lee, op. cit.). Unless otherwise specified, DNA template concentration was 1 nM, and the reactions incubated for 2.5 hrs at 25° C. NiCl₂ or CoCl₂ were added to the translation extracts of ColE9 or ColE7, respectively, to a final concentration of 1 mM, followed by 10 minutes incubation at room temperature or over-night at 4° C. The translation extracts were then mixed at various nuclease:inhibitor ratios (1:1-1:4). The DIG-Biotin DNA substrate was added to 5 nM concentration, and the digestion reactions incubated at 25° C. for various time periods. Aliquots at different time points were quenched by 33-fold dilution in B&W buffer (1M NaCl, 10 mM Tris, 25 mM EDTA, 15 mM EGTA, pH 7.4). 200 μl of quenched solutions were added to streptavidin-coated 96-well plates (Nunc) and incubated for 1 hr. The plates were rinsed 3 times with twice-concentrated B&W and PBS/T/BSA (PBS supplemented with 0.5% Tween20 and 0.2% BSA). 200 μl of a 1:1500 dilution in PBS/T/BSA anti-DIG-HRP conjugated antibody (Jackson) was added for 1 hr. The plates were rinsed 3 times with PBS/T and once with PBS, 200 μl of TMB substrate (Dako) were added, and the O.D. at 405 nm measured.

The ColE9 gene was translated in cell-free extracts at 2 nM, for 2.5 hours at 25° C. The DIG-Biotin DNA substrate was added to 100 μl of these extracts on ice, to a final concentration of 5 nM. The reaction mixture was added to 1 mL of ice-cold oil mix comprised of 4.5% (w/w) Span80, 0.5% (w/w) Tween80 in light mineral oil (Sigma), placed in 2 mL cryotube (Corning). This emulsion mixture was kept in ice-water bath and homogenized for 5 minutes at 8000 RPM in IKA (Ultra Turrax T25) homogenizer equipped with a disposable shaft (OmniTip). The emulsions were then transferred to 25° C. Micelles systems were prepared by adding 250 mM NiCl₂ water solutions to 250-fold excess (v/v) of light mineral oil containing 7.5% (w/w) Span80 and 2.5% (w/w) Tween80. The mixture was extensively mixed (hard vortex followed by shaking), to obtain a clear solution. A precipitate would sometimes appear after longer incubations yet the clear supernatant was used in all cases to mediate the metal ion delivery. Merging the micelles with the aqueous droplets of the water-in-oil emulsion was carried out as follows: 500 μl of NiCl₂ micelles solutions were added to the emulsion, followed by gentle mixing and 2-16 hr incubation at 25° C.

To break the emulsion and isolate the genes, the emulsion was spun down at 10600 g for 5 min. The oil phase was removed and 400 μl of B&W buffer supplemented with 40 μgr/ml yeast RNA, 25 mM EDTA and 15 mM EGTA, were added, followed by 1 ml of water-saturated ether. The tube was vortexed and the ether phase removed. The aqueous phase was rinsed twice with ether, and traces of ether removed by SpeedVac drying for 5 mins. The concentration of the DNA substrate in the samples was subsequently determined by nuclease activity assay as described above.

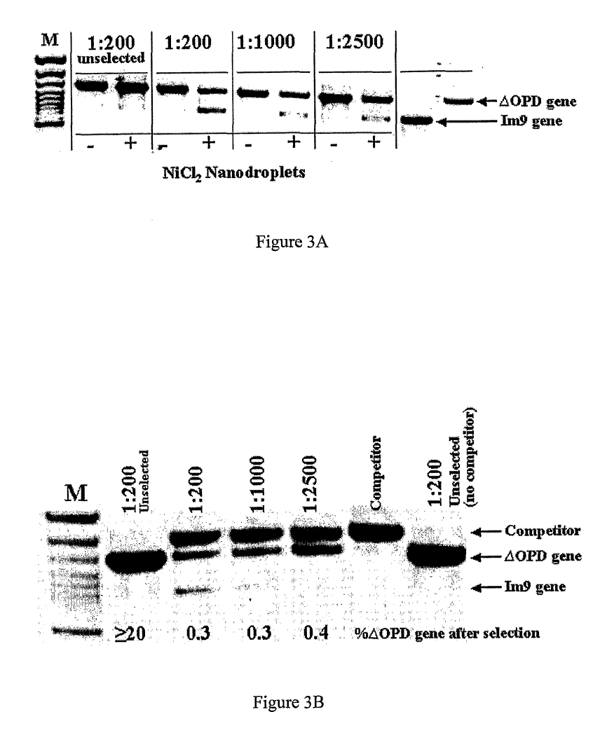

The Model Selections used is as follows: 100 μl of ice-cold cell-free extracts containing 400 pM of the ΔOPD gene and various concentrations of the Im9 gene (2 pM, 0.4 pM or 0.16 pM; corresponding to 1:200, 1:1000 and 1:2500 ratios of Im to ΔOPD), were supplemented with 10 μl of extract, in which the ColE9 gene was translated (3 nM template DNA, 4 hrs at 25° C.). The extract mixture was emulsified as above. The emulsion was incubated for 4 hrs at 25° C. to allow the translation of the ΔOPD and Im9 genes. 500 μl of NiCl₂ micelles solution were added, and the mixture incubated for 16 hrs at 25° C. The emulsions were broken as above, and the ether-rinsed aqueous phases were added to 200 μl of B&W buffer plus 8 μl of M280 streptavidin-coated magnetic beads (Dynal), and incubated for 1 hr. The beads were rinsed 3 times with twice-concentrated B&W and 3 times with 5 mM Tris-HCl pH 8, and then resuspended in 811 PCR buffer (16 mM (NH₄)₂SO₄, 67 mM Tris-HCl pH 8.8, 0.1% Tween20). For PCR amplification, 2 μl of bead suspensions were diluted 10-fold in PCR buffer corresponding to a 105 dilution of the original DNA mix before selection, and amplified. Concomitantly, 0.4 pM of Im9 genes were similarly diluted and separately amplified. PCRs were performed with BioTaq (BioLine) for 30 cycles (95° C. 0.5 min; 63° C., 0.5 min; 72° C. 1.5 mins) using primers LMB2-6 and PIVB6. The PCR products were analyzed on 1% agarose-TAE gels with DNA marker GeneRuler™ 100 bp ladder (Fermentas). Competitive PCR (FIGS. 3B and 4) was preformed with the DNA solutions recovered from the emulsions described above. These were mixed with equal volumes of a competitor gene (an 1320 bp insert cloned into NcoI/SacI sites in pIVEX) at a concentration of 4 pM (corresponding to 1% of the initial concentration of ΔOPD gene used in selection). 1 μl of this DNA mixture was diluted 100-fold in PCR buffer, and amplified in 20 μl PCR reactions using primers LMB2-6 (Bc) and PIVB6 (Fo). The reactions were cycled 30 times, and the PCR products analyzed on 1% agarose-TAE gel.

Im9 gene libraries were prepared as follows: Randomization by error-prone PCR was based on previously described protocols. Briefly, 1 ng of pIVEX-Im9 DNA was amplified in PCR reactions containing NTPs (200 μM in total) at 1:5 or 1:10 ratios of AC:TG, supplemented with 250 μM MnCl₂, using the LMB2-9 and pIVB10 primers (25 cycles: 95° C. 0.5 min; 53° C. 0.5 min; 72° C. 1.5 mins in 1:5 bias, and 2 mins in 1:10 bias). The PCR product was virtually-cloned and amplified as above. A fraction of the ligated pIVEX plasmid was transformed into DH5α cells and several individual clones were sequenced to show a mutation rate of 1.14% and 1.64% in the 1:5 and 1:10 bias libraries. This percentage corresponds to an average of 3 and 4 mutations per gene (for the 1:5 and 1:10 bias libraries, respectively). Of the total mutations, 50% and 75%, bias 1:5 and 1:10 respectively, were transition mutations, and the rest transversion mutations, and, 20% and 30% were synonymous mutations.

DNA shuffling was performed using exiting methods. Briefly, the pool of genes coming from the 5^{th} round of selection was mixed with the wild-type Im9 gene at 1:1 ratio. The DNA was digested with DNaseI. DNA fragments of 75-125 bp length were gel-purified and PCR-assembled (10 ng DNA fragments; 94° C. 0.5 min, and then 35 cycles composed of a temperature gradient of 65° C.-41° C., 1.5 mins at each temperature followed by 45 seconds at 72° C.). The PCR product was captured on M280 streptavidin coated magnetic beads (Dynal) as known in the art (e.g. U.S. Pat. No. 4,921,805). The beads were rinsed with twice-concentrated B&W buffer and PCR buffer. The bound DNA was PCR-amplified using primers LMB2-9, pIVB10 (18 cycles; 95° C. 0.5 min, 53° C. 0.5 min, 72° C. 1 min), digested by SphI and PstI (restriction sites upstream and downstream to NcoI and SacI sites, respectively), and virtually-cloned into the pIVEX vector as described above.

Library selections were done essentially as the model selections described above. Each round was performed under changing DNA concentration, time and temperature of incubation (following the metal ion delivery by micelles) as specified in FIG. 5. After the first round of selection, the 1:5 and 1:10 bias libraries showed the same level of DNA survival and were combined into one library for the subsequent rounds of selection.

In vivo protection assays were performed essentially as described (Kleanthous et al., 2004, J Mol Biol 337, 743-59). Briefly, the newly-evolved, and wild-type, Im variants were cloned into the IPTG-inducible expression plasmid pTrc99a (Pharmacia Biotech), and transformed to E. coli JM83 cells (kindly provided by Kleanthous). Cells were grown as lawns on Ampicilin-LB agar plates without, or with IPTG (0.05, or 1 mM), and spotted with ColE7 at different concentrations. Cell death was visualized in the form of plaques after ON incubation, and the lowest concentration of ColE7 at which there was no formation of plaques was recorded (Table 3).

Cell-free translation allowed expression of three different ColE genes to yield enzymatically active nucleases. This provided a mean of selecting immunity protein inhibitors in a completely in vitro fashion, and of circumventing the need to isolate the ColE protein after co-expression with their cognate immunity protein. ColEs were activated in cell-free extracts by addition of cobalt or nickel ions, but not by magnesium, as previously reported (Pommer et al., 1998, Biochem J 334(Pt 2):387-92 and Pommer et al., 1999, J Biol Chem 274:27153-60). It appears that these metals stabilize the structure of ColEs, a role that is suggested to be fulfilled also by immunity protein binding.

A new immunity protein variants was selected out of a library derived from the Im9 gene. The unselected library exhibited almost no inhibition towards either ColE9 (the cognate nuclease of Im9) or ColE7 (the target of selection). The selection pressure was modulated through the rounds of selection to attain both high recovery and enrichment. By the 5^{th} round of selection, individual variants were identified that showed some convergence towards specific sequence changes, which were then observed by the end of the selection process (Round 8).

After eight rounds of selection, the inhibition activity of the best variants was still much lower than that of wild type Im7, indicating that the evolutionary transition from Im9 activity into Im7 activity is clearly incomplete. Due to the need to express and purify colicins, and the very long dissociation half-lives of their complexes, the affinity of the newly-evolved Im proteins is yet to be measured. Thus, to provide support for our in vitro assays, the in vivo protection assays applied by Kleanthous and coworkers was followed. These assays correlate the affinity constants of Im protein variants with the degree of protection against ColE toxicity in vivo. The protection generally varies between K_{d} values that are >10⁻⁸ M (0% protection) and K_{d}<10⁻¹¹ M (100% protection). These protection assays show a dramatic increase in the ability of the selected Im9 variants to inhibit ColE7 (Table 3). Table 3 lists, for each Im variant, the minimal ColE7 concentration (in Molar) at which full protection was observed. Wild-type Im9, which binds ColE7 with a K_{d} of 3.8×10⁻⁸ M, exhibited protection only at the lowest ColE7 concentrations (0.3×10⁻¹⁰ M, at the highest Im9 expression levels; Table 3). The best 8^{th} round variants (#4, 7 & 8) protects up to ColE7 concentrations of 10⁻⁴M to 10⁻⁹M depending on the expression level of the Im proteins. The in vivo protection assays therefore suggest that these variants exhibit K_{d} values in the range of 10⁻¹⁰ to 10⁻¹¹ M.

TABLE 1
Activity of DNase ColE9 in cell-free extracts
% of DNA survival

| Sample | Bulk assay^{a} | Emulsion sample |
|---|---|---|
| Extract | 100^{b} | 100 |
| Extract + ColE9 | 59.4 | 25 |
| Extract + ColE9 + Ni⁺² micelles | ≦5 | 1.5^{c} |
| Extract + ColE9 + Im9 + Ni⁺² | 100 | n.d. |

^{a}Assays in bulk solution were performed by incubation for 15 mins of the DIG-biotin labeled DNA substrate with extracts expressing ColE9, with or without nickel ions, at 25° C.
^{b}In emulsions composed of extract with no ColE9, the percentage of surviving DNA was essentially identical with or without the addition nickel ions.
^{c}In vitro assay. DNA survival was as low as 0.01% when higher volumes of cell-free extracts expressing ColE9 were added (see FIG. 4).
n.d.—not determined

TABLE 2
Sequence and inhibition activity of the 8 round in vitro evolved immunity proteins
Immunity protein binding and selectivity-determining residues^{a,b}

| | ‘Variable specificity region’ and other residues | | | | | | | | | | | | ‘Conserved hot spot’ residues | | | |
|---|---|---|---|---|---|---|---|---|---|---|---|---|---|---|---|---|
| | Position (Im9 numbering)/% ColE7 inhibition^{c} | | | | | | | | | | | | | | | |
| | 24 | 26 | 27 | 28 | 30 | 33 | 34 | 37 | 38 | 41 | 42 | 50 | 51 | 54 | 55 | 56 |
| Im9/0 | Asn | Asp | Thr | Ser | Glu | Leu | Val | Val | Thr | Glu | Glu | Ser | Asp | Tyr | Tyr | Pro |
| Variant 1^{d}/33 | Asp | | Ala | Thr | | | | | | | | | | | | |
| Variant 7/69 | | | | Thr | | | Asp | Ile | | | | | | | | |
| Variant 4/72 | Asp | | Ala | Thr | | | Asp | Ile | | | | | | | | |
| Variant 6/66 | Asp | | Ala | Thr | | | Asp | | | | | | | | | |
| Variant 8/97 | Asp | Asn | Ala | | | | Asp | Ile | | | | | | | Trp | |
| Corresponding | Lys | Asn | Val | Ala | Glu | Leu | Asp | Leu | Glu | Val | Lys | Thr | Asp | Tyr | Tyr | Pro |
| position in Im7 | | | | | | | | | | | | | | | | |

^{a}The table lists all residues previously implicated in complex formation of both ColE9-Im9 and ColE7-Im7, as well as Im9 residues in which highly conserved mutations were found in the newly evolved variants (e.g., residues 27 and 28.
^{b}Additional mutations observed in the newly-evolved variants in residues that are, in most likelihood, not involved in colicin binding are: Variant #7, Glu2Gly, Lys57Glu; Variant #A, Ser6Gly. Phe83Leu; Variant #4, Lys57Glu; Variant #6, Met43Thr; Variant #8, Ser6Arg.
^{c}Inhibition of the DNase activity by the newly-evolved variants, wild-type Im7 and Im9, was determined by bulk nuclease activity assay. The reaction mixtures were incubated at 33° C. in the presence of the DNA substrate for 5 min. Under these assay conditions, 100% inhibition was observed with cognate pairs and 0% with non-cognate.
^{d}Variant 1 and 7 were isolated from Round 8 performed under low stringency conditions; all other variants were isolated from the high stringency selection.

Although the Im variants were selected under conditions that are quite different than those prevailing in living E. coli cells, the selection pressure in the emulsion droplets led to an increased in vivo potency (Table 3). Another notable feature is the similarity in sequence changes between the newly-evolved Im variants and their natural counterparts.

All the meaningful sequence changes occurred at the ‘variable specificity region’ around Loop I and Helix II, while the ‘conserved hot spot’, at the region Helix III (including Asp 51, Tyr54 and Tyr55 of Im9) remained essentially unchanged (Table 2).

TABLE 3
The inhibitory activity of the in vitro evolved immunity
proteins in an in vivo protection assay.

| Im Variant | w/o IPTG | 0.05 mM IPTG | 1 mM IPTG |
|---|---|---|---|
| Im7 | ≧10⁻⁴ | >>10⁻⁴ | >>10⁻⁴ |
| Clone 8 | 0.3 × 10⁻⁹ | 0.3 × 10⁻⁷ | >10⁻⁴ |
| Clone 4 | 10⁻⁹ | 0.3 × 10⁻⁷ | >10⁻⁴ |
| Clone 7 | 0.3 × 10⁻⁹ | 1 × 10⁻⁸ | >10⁻⁴ |
| Clone 6 | 0.3 × 10⁻¹⁰ | 1 × 10⁻⁹ | 0.3 × 10⁻⁸ |
| Clone 1 | <10⁻¹¹ | 10⁻¹¹ | 10⁻¹¹ |
| Im9 | 10⁻¹¹ | 0.3 × 10⁻¹¹ | 0.3 × 10⁻¹⁰ |
| ΔΔOPD | n.d | n.d | 10⁻¹¹ |

In view of the completely random nature of the mutations in the unselected library, these results confirm the proposed mechanism of ‘dual recognition’, as well as the hypothesis regarding the routes by which colicin-immunity interaction diverged during natural evolution. Thus, the ‘conserved hot spot’ appears to provide a common motif and a starting point for the evolution of new pairs, whereas divergence is mediated only by changes in the variable region (Helix II) of the immunity protein. The role of the ‘conserved hot spot’ in providing an initial of cross-reactivity, and thereby a starting point for the evolution of new pairs is analogous to the possible role of enzyme promiscuity (or substrate ambiguity) in the evolution of new enzyme functions.

### Example 2

### Expression and Activation of ColEs in Emulsion Compartments

Directed evolution of nuclease inhibitors is ideally performed in vitro since all nucleases are toxic to living cells. We found that both the ColE7 and ColE9 genes translate efficiently in vitro, namely in cell-free extracts, and can be then activated by addition of divalent metals ions (Co⁺² for ColE7, and Ni⁺² for ColE9). The In vitro translated Im proteins were also active, since addition of cell-free extracts in which the Im7 or Im9 genes were translated, completely blocked the activity of the cognate ColE (Table 1). (For brevity, we refer to cell-free extracts in which a given gene was transcribed and translated, e.g., Im7, as ‘Im7 cell-free extract’).

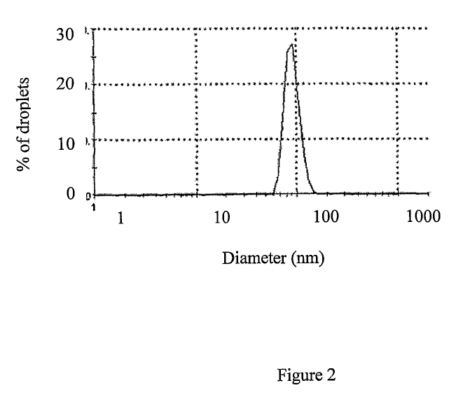

Micelle solutions were prepared by adding aqueous solutions of bivalent salts (e.g., NiCl₂, CoCl₂) to a 250-fold volume excess of mineral oil containing 7.5% Span80 and 2.5% Tween80. The mixture was shaken extensively until a clear solution has been obtained. The clear supernatant of a 250 mM NiCl₂ micelles solution was analyzed by the light scattering HPPS instrument (Malvern Instruments). Size distribution analyzed either by number, and by volume, gave a mean droplet diameter of ˜100 nm (0.1 μm), indicating swollen micelles or micelles with >30-fold smaller diameter then the emulsion droplets (FIG. 2). The NiCl₂ micelles solutions were then added to emulsions containing ColE9 cell-free extracts and 0.5 nM DNA substrate. The emulsions were incubated to allow DNA digestion to proceed, and then broken. The amount of undigested DNA substrate was determined by a nuclease activity assay and competitive PCR. In the absence of metal ions, DNA digestion was incomplete even after long incubations. However, a dramatic increase in the level of DNA digestion was observed following the addition of the micelles nickel solution indicating that the nickel ions have indeed reached the aqueous droplets and activated the ColE9 (Table 1, above). DNA survival was even lower when higher volumes of ColE9 cell-free extracts were added as demonstrated in FIGS. 3 and 4.

The addition of the micelles solutions had no significant effect on the stability or size distribution of the emulsion droplets.

Using the micelles delivery system described above, genes encoding Im9 could be enriched from a large excess of ΔOPD genes encoding a protein with no inhibitory activity. The Im9 and ΔOPD genes were amplified from a construct carrying a T7 promoter, and labeled with biotin at their 5′ end. The ColE9 genes were translated in 10 μL of cell-free extract, and this extract (‘ColE9 cell-free extract’) was added to fresh extract containing mixtures of the Im9 and ΔOPD genes in various ratios. The extract was compartmentalized by emulsification to give, on average, ≦1 gene per compartment. The emulsions were incubated to complete the translation of the Im9 and ΔOPD genes within their respective compartments, and the nickel chloride micelles were added to allow ColE9 activation and DNA digestion. Only in half of the samples ColE9 was activated by addition of NiCl₂ micelles (labeled as “+micelles”). The emulsions' structure was brought to coalescence, the DNA was captured from the aqueous phase onto streptavidin-coated magnetic beads and amplified by PCR. The level of survival of the gene in excess (ΔOPD) was determined by competitive PCR. The PCR products were analyzed by agarose gel electrophoresis. The intensity ratio, between the ΔOPD and the competitor band, corresponds to the percentage of ΔOPD genes that survived the ColE9 digestion and is indicated in bold. The results of these selections indicated ˜100-fold enrichment for the genes encoding the inhibitor Im9 over the ΔOPD genes (FIG. 3A). Starting from a ratio of 1:200, 1:1000 and up to 1:2500 Im9 to ΔOPD genes in fresh extracts, the compartmentalized selections gave a mixture of these genes at ratios of ˜1:3 down to about 1:20. No enrichment was observed without the addition of the nickel ion micelles solution.

The recovery of Im9 genes surviving the compartmentalized selection process was estimated by competitive PCR against a third gene of a different length (FIG. 3B). This experiment indicated that, under this selection pressure, ˜0.3% of ΔOPD genes had survived, regardless of the initial concentration of the Im9 gene. The ratio of ΔOPD:Im9 gene after selection is ˜3:1, and the fraction of Im9 genes that survived the selection is therefore ˜0.1%. Since the initial fraction of Im9 genes before selection was 1:200 (0.5%), the recovery of the Im9 genes is ˜20%. Thus, the described selection procedure exhibits effective recovery of the ‘positive’ genes (20%) and reasonable enrichments (>100 fold). Enrichment is limited primarily by a sizeable fraction of ‘false positives’ (˜0.3%) due to genes that escape ColE9 digestion despite the absence of an inhibitor.

In the experiment, the results of which are provided in FIG. 4, various volumes of cell free extracts (10 μl-40 μl), in which either the ColE7, or ColE9, genes were translated at 4 nM, mixed with aliquots of 100-70 μl of fresh extract containing 100 μM of the Im9 genes (total volume of 110 μl) and emulsified. The emulsion was incubated to allow the translation of Im9 gene and the colicin DNases were then activated by micelles delivery of metal ions (24 hrs at 25° C. followed by 30 mins at 30° C.). The emulsions were broken, and biotinylated Im9 genes were captured on beads. The level of survival of the Im9 genes was determined by competitive PCR (see experimental section). The competitor gene was added at amounts equivalent to 10%, 1% and 0.1% of the initial Im9 gene concentration. The products of the competitive PCR were analyzed on agarose gel and quantified by densitometry (Image Gauge v3.0). The ratio between the two the competitor and the Im9 gene provided an estimate to the survival of the selected Im9 gene. The results are summarized in Table 4.

TABLE 4
Selectivity and stringency of the selection pressure
Nuclease IVT (μl)

| 10 | | | 20 | | | 40 | | |
|---|---|---|---|---|---|---|---|---|
| % Remaining DNA | | | % Remaining DNA | | | % Remaining DNA | | |
| Cognate | Non-cognate | Enrichment | Cognate | Non-cognate | Enrichment | Cognate | Non-cognate | Enrichment |
| 8.5 | 0.7 | 12 | 12.4 | ˜0.02 | >500 | 7.2 | ˜0.013 | >500 |

The survival of Im9 gene emulsified with a cognate DNase (ColE9) appears to be ˜10%, regardless of the amount of ColE9 added. However, in the presence of the non-cognate ColE7, survival of the Im9 gene goes down, from 0.7% to 0.013%, as the volume of the ColE7 extract is increased. The ‘enrichment’ corresponds to the ratio of survival of the Im9 gene in the presence of the cognate vs. non-cognate colicin (ColE9 and ColE7, respectively). Indeed, FIG. 4 indicates that much higher enrichments (≦500-fold enrichment, and 0.01% of undigested DNA) were obtained with this system when the efficiency of DNA digestion was improved by adding higher volumes ColE9 cell-free extracts.

For an evolutionary process to succeed, the selection pressure must change during its course. At the beginning, the selection pressure should be low to allow survival of all genes that encode a protein with the desired activity, be it low or high, so that no or little diversity is lost (high recovery). As the evolutionary process progresses, the selection pressure needs to be increased to allow genes encoding proteins with the highest activity to compete, thus leading to convergence rather then divergence of sequence (high enrichment). The selection system described here offers several ways by which the selectivity and stringency of the selection can be tuned.

An effective way of increasing selection pressure is by changing the volume ratio between the ColE cell-free extract, and the fresh extract in which the immunity genes are translated. This increases the selection pressure in two ways: first, by increasing the concentration of the ColE nuclease; and second, by decreasing the translation levels of the immunity protein. In this way, the recovery of genes encoding an inhibitor with low affinity (e.g., a non-cognate immunity protein) can be easily tuned over a 50-fold range (from 0.7% down to 0.012%; FIG. 4). FIG. 4 also shows the selectivity of the selection since, in oppose to the low-recovery of non-cognate immunity genes, ˜10% of the cognate genes survive. The selection pressure can be further modulated by changing the incubation temperature, and time, with the nickel ion micelles. The very broad dynamic range of this selection system allowed us to control the threshold of the inhibitor's affinity, and to perform library selections as described below.

### Example 3

### Evolution of Im9 into a ColE7 Inhibitor

We aimed at reproducing in the test tube the evolution of a new specificity in an existing member of the immunity protein family. The diversification of natural immunity proteins is attributed mainly to high mutation rate during replication and to recombination. Random mutagenesis and homologous recombination were also used to diversify the Im9 gene for in vitro evolution, using error-prone PCR and DNA shuffling. Error-prone PCR in the presence of biased nucleotide ratios and manganese chloride was calibrated to an average mutation rate of 2 or 3 mutations per gene. This mutation rate gave the best enrichment and recovery. A library with higher mutation rate (13-20 mutations per gene) showed no enrichment after four rounds of selection. Additional mutations had accumulated during the numerous PCR cycles used to amplify the surviving genes after each round of selection (an average of 6 mutations per gene was observed after rounds 5 and 8 of the selection). The libraries of Im9 genes were selected for inhibition of ColE7. Following each round of selection, progress was monitored by competitive PCR to assess the percentage of surviving genes, and by assaying the inhibition activity of the pool of genes towards ColE7 (FIG. 5).

The selection pressure was gradually increased, starting at a low selection pressure aimed at getting high recovery of genes (20 μl ColE7 cell-free extract, 50 pM selected DNA, and 0.5 hr incubation at 30° C.). As the evolutionary process progressed, we significantly increased the selection stringency (34 μl ColE7 cell-free extract, 25 pM selected DNA, 5 hrs incubation at 37° C., in the last round of selection; FIG. 5). By the fifth round, inhibitory activity of ColE7 could be clearly observed. The pool of genes was cloned in E. coli, and sequencing of positive clones revealed several beneficial mutations at the ‘variable specificity region’ of Im9, along side mutations that seemed potentially damaging (e.g., a Ser to Pro, at position 65 in the middle of a helix). Backcrossing and homologous recombination of the selected clones were performed, by mixing the pool of genes from Round 5 with wild type Im9 at 1:1 ratio, and performing DNA shuffling. The shuffled library was subjected to 3 additional rounds of selection. The last round (Round 8) was performed at high stringency (5 hrs incubation at 37° C.) as well as low stringency (1 hr incubation at 37° C.).

The pool which survived the higher stringency conditions exhibited ˜50% inhibition of ColE7's DNase activity under conditions that yield 0% inhibition by Im9, and 100% by wild-type Im7 (FIG. 5), whereas the pool of genes recovered from the less stringent selection condition (1 hr incubation) showed ˜4 fold less activity. Both pools of genes were cloned in E. coli. Individual clones were amplified and the resulting DNA translated in cell-free extracts and assayed for inhibition of ColE7 and E9. About half of the tested clones were found to effectively inhibit ColE7 to various degrees (Table 2). As expected, several mutations in the ‘variable specificity region’, which appeared in separate clones from Round 5 (e.g., Val 34Asp and Asp26Asn) were combined in single Round 8 clones. In addition, several mutations that we suspected to be neutral or harmful, disappeared: these include, Leu3Pro, Thr20Lys, Ser35Pro, Thr38Glu, Lys57Ser, Ser65Pro, Ser65Glu and Lys80Glu. The ability to modulate the stringency of selection was also manifested in the properties of individual immunity variants. Variants obtained from Round 8 performed at low stringency, exhibited distinctly lower inhibitory activity (in average ˜3 fold difference in activity, e.g., Variants 1, Table 2) than those isolated from the high stringency selection (66-97%).

The increased ability of the in vitro evolved variants to inhibit ColE7 was confirmed by an in vivo protection assay. Briefly, agar lawns of cells expressing the wild-type and newly-evolved Im variants were grown and the plates were spotted with the ColE7 toxin complex at various concentrations. Cell death was visualized in the form of a plaque after ON incubation, and the highest concentration of ColE7 under which no cell death was apparent was recorded for each variant (Table 3, above). As previously observed (Kleanthous, op. cit.), these concentrations change with the level of Im protein expression as dictated by the level of IPTG induction. The 8^{th} round variants show a marked ability to protect against ColE7 at concentrations that are 10² (no IPTG) up to 10⁵ (1 mM IPTG) higher than Im9. The order of the in vivo protection capabilities roughly correlates with the order of inhibition seen with the in vitro assays (Table 2), with Variant #1 being the poorest, and variants #4, 7 and 8 being the most potent.

Sequence analysis of Round 8 clones (Table 2, above) showed convergence into residues at the ‘variable specificity region’ of Im9, two of which (Asn26, Asp34) appear in wild type Im7 (26 and 35 by Im7 numbering). These two residues are known to significantly contribute to binding of Im7 to CoE7, via hydrogen and electrostatic bonds. The mutation Val34Asp seems to be the most significant source of improved ColE7 inhibition, it appears to play a key role as indicated by the much lower inhibition exhibited by variant #1 that does not carry it. Other changes in the sequence of Im9 are characterized by the addition of negative charges (Asn24Asp, Lys57Glu), which is in agreement with Im7's specificity residues being of charged nature, compared to the more hydrophobic Im9. In addition, conserved changes in residues 27 and 28 (Thr27Ala and Ser28Thr) were observed in most of the selected clones. The net effect of these substitutions, from polar into hydrophobic, is reasonable since these residues are Ser and Thr in wild-type Im9, and Val and Ala in Im7. We presume that the mutations observed in residues 24, 27 and 28 have a smaller effect on activity as no significant change in inhibition was observed between variant that carry these mutations (e.g., variant 7) and variants that do not (e.g., variants 4 and 6). The high frequency of these mutations within the selected variants can be attributed to their linkage with other beneficial residues (these mutations seem to appear in clones of Round 5, together with either Val34Asp or Asn24Asp), or simply due to the haphazard fixation of neutral mutations during the evolutionary process. Other mutations do not pose a dramatic change from wild type residues (e.g., Val37Ile) yet their conservation suggests that they are of relevance. The rest of the mutations observed in the newly evolved Im variants are in areas that are remote from the colicin binding site region, and also vary from one variant to another are listed in footnote b of Table 2.

Only one selected variant appears to have a mutation in the ‘conserved hot spot’ in Tyr55 that confers a considerable degree of colicin binding energy in all immunity proteins (Tyr55Trp, variant 8). The activity assays (Table 2) and data by others on the same mutation, suggest that this mutation does not lead to significant loss of binding affinity.

### Example 4

### Selection for Affinity of the Evolved Variants

Affinity measurements of the evolved variants, performed at the laboratory of Prof. Colin Kleanthous (York, UK), indicate an increase of affinity of >10⁴-fold towards their selection target ColE7, and show the wide dynamic range of the selection method of the invention. The results are summarized in Table 5.

TABLE 5
Affinity measurements.

| | kₒₙ | k_{off} | K_{d} |
|---|---|---|---|
| Complex | (×10⁸ M⁻¹ s⁻¹) | (s⁻¹) | (M) |
| ColE7-Im7 | 7.6 | ˜10⁻⁵ | ˜10⁻¹⁴ |
| ColE7-Im9 | 0.96 | 5.3 | 5.6 × 10⁻⁸ |
| ColE7-Evolved Im variant#8 | 8.3 | 3.2 × 10⁻³ | 3.85 × 10⁻¹² |
| ColE9-Im9 | 0.78 | nd | <10⁻¹⁴ |
| ColE9-Evolved Im variant#8 | 1.18 | 2.6 × 10⁻³ | 2.19 × 10⁻¹¹ |

Albeit, it can be seen that the affinity of the evolved variants towards their original target DNase (ColE9) is still very high and is comparable to their affinity towards the selection target (ColE7).

Directed evolution faces a very common bottleneck. It can readily modify an existing protein function and improve it by many-fold (e.g., increase the binding of an Im towards a new target colicin). But dramatically reducing, let alone eradicating, the protein's original function (e.g., the binding of an engineered Im to its native colicin) is constantly proving a Herculean task. This is no coincidence, nor a technical flaw. We have shown that while the promiscuous functions of proteins are subjected to large changes (either increase or decrease) in response to few, or even one mutation, their native functions tend to remain largely unchanged. Thus the native function is resistant, or robust, towards mutations in the very same active site that mediates the promiscuous functions. This seems like a generic property of proteins that stems from the fact that their native functions have been constantly under selection, thus evolving a high degree of robustness, while the promiscuous functions (e.g., the cross-reactivity of Im7 with ColE9) that are latent and were never under selection, exhibit high plasticity.

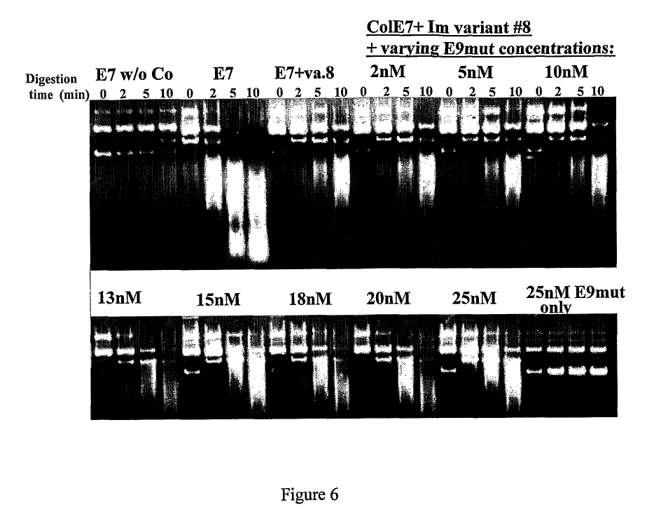

To overcome this obstacle, the inventors of the present invention developed a novel selection system that enriches for higher affinity as well as selectivity—i.e., for variants that bind and inhibit the target colicin, and show a marked decrease of binding of other ColE nucleases. The basis will be colicin variants with a mutated active site histidine (e.g. His103Ala or His127Ala E9 DNase) which has no DNase activity yet binds Im protein with affinity and selectivity that is essentially identical to wild-type colicin. During selection in the in vitro compartments, these mutated ColEs can compete with the target ColE (that does posses DNase activity) in binding any variant that is not sufficiently selective, and drive the enrichment of variants with higher affinity and selectivity. Indeed, it can be seen in FIG. 6 that the inhibition activity of the evolved Im9 variant #8 that binds ColE7 with relatively high affinity (K_{d}^{ColE7}=3.8×10⁻¹² M) but remains highly cross-reactive towards ColE9 (K_{d}^{ColE9}=2.2×10⁻¹¹ M) (see Table 5 above), significantly decreases with increasing concentrations of the ColE9H127A mutant. In contrast, the inhibition of ColE7's DNase activity by wild type Im7 (that is highly selective towards ColE7 and barely bind ColE9; K_{d}^{ColE7/Im9}>10⁻⁵ M) is not affected by the ColE9H127A mutant even at the highest concentration tested (100 nM). In this experiment (FIG. 6), ColE7's digestion activity was assayed in a plasmid-nicking assay as known in the art (e.g. Terry et al., J. Virology 62:2358-2365, 1988). The reactions contained 10 nM E7 activated by cobalt ions, in the presence of 45 nM evolved variant 8 and increasing concentrations of the ColE9H127A mutant (E9mut). An aliquot of the reaction was analyzed on agarose gel at 4 different time points. The inactive E7 (E7 w/o Co) and the E9mut alone show no significant digestion activity. In the presence of variant 8, it can be seen that, ColE7's activity is almost completely blocked (E7+va8). However, inhibition decreases with the increasing concentration of the E9mut (2 nM-25 nM), to a stage where almost none is observed. In contrast, the inhibition of ColE7's DNase activity by wild type Im7 is not affected by the ColE9H127A mutant even at the highest concentration tested (≦100 nM).

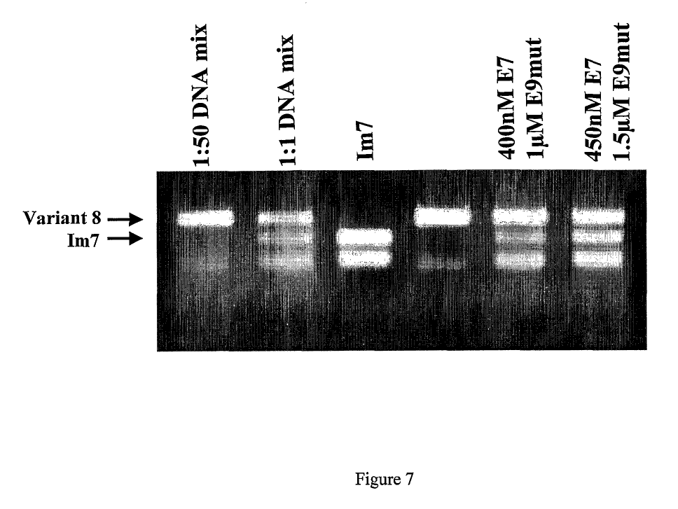

Further, this selection system can enrich for the highly selective wild-type Im7 (K_{d}^{ColE7}=7.9×10⁻¹⁶ M; K_{d}^{ColE9}=5.6×10⁻⁸ M) from a large excess of an in vitro evolved Im9 variant #8 which is cross-reactive towards ColE9 (FIG. 7). Selections were performed essentially as known in the art with the exception that the ColE9H127A mutant (E9mut) was added to some of the samples. The Im7 and evolved variant 8 (va.8) genes were mixed at 1:50 ratio respectively (DNA mix). The DNA mix was emulsified together with a cell free extract containing 400 nM E7+1 μM E9 H127A mutant (E9mut), or 450 nM E7+1.5 μM E9mut. After translation of the genes and E7 activation the emulsion was broken, and the surviving genes captured on magnetic beads and PCR-amplified. The amplified DNA was digested with DpnII which selectively digests Im7 but not va.8. The ratio of Im7:va8 after selection is estimated as 1:10 to 1:1 indicating a 5 fold and a 50 fold enrichment factor at the lower, and higher, ColEs concentrations, respectively. As control, the gel indicates the bands resulting from digestion of the original 1:50 DNA mix, a 1:1 DNA mix, and each of the genes on its own (Im7, and variant8). Note that, the lower band on the gel appears in all digestion reactions, and results from the digestion at a common site outside the open reading frames of both genes. Preliminary results indicate that selections of libraries derived from variant 8, performed in the presence of ColE7 plus the ColE9 H127A mutant, yield mutants with dramatically lower affinity towards ColE9.

### Example 5

### A Novel Use of Abil® EM90 Emulsion-Optimized Single Molecule PCR

For preparing Abil® EM90 emulsions the water phase (100 μl for a single emulsion) was composed of 1.7 mM MgCl₂, 1 μM of each primer, 0.25 mM of each dNTP, 0.5 mg/ml BSA, ˜10⁸ molecules of template DNA (since the emulsification conditions lead to ˜10⁹-10¹⁰ water droplets per emulsion, this DNA concentration ensures that one DNA molecule will be present per droplet), and 12 units of BioTaq (Bioline) in 1× BioTaq buffer (16 mM (NH₄)₂SO₄, 67 mM Tris-HCl (pH 8.8), 0.01% Tween 20).

The emulsion was prepared by slowly adding the ice-cooled water phase (100 μl total in 7 μl aliquots) to 900 μl of the ice-cooled oil phase (2% Abil® EM90, 0.05% Triton X-100 in mineral oil) in a Costar Tube (Corning #2051) while stirring with a magnetic stirring bar (1400 RPM). After addition of the water phase (which takes 2 min) the emulsion is stirred for another 5 min.

The emulsion was transferred in 60 μl aliquots to 0.2 ml thin-wall PCR tubes. The PCR reactions were done on an Eppendorff Mastercycler with a temperature ramp of 0.3° C./sec as follows: 2 min at 94° C. for initial DNA denaturation, followed by 32 cycles of 94° C. for 30 sec, 50° C. for 30 sec and 72° C. for 2 min, and a final incubation at 72° C. for 10 min.

The emulsion was then subjected to PCR with a temperature ramp of 0.3° C./sec as follows: 2 min at 94° C. for initial DNA denaturation, followed by 32 cycles of 94° C. for 30 s, 50° C. for 30 sec and 72° C. for 2 min, and a final incubation at 72° C. for 10 mm.

After the PCR reaction was completed, all aliquots were combined and centrifuged at 5000 RPM for 5 min at 4° C. Most of the upper oil phase was removed and the remaining emulsion was broken by addition of 100 μl of 50 mM Tris-HCl (pH 7.9), 10 mM EDTA and 1 ml of Ether. The mixture was then extracted three times with Ether and finally the remaining Ether was removed by Speed Vac for 40 min. The PCR reaction can now be analyzed by agarose electrophoresis.

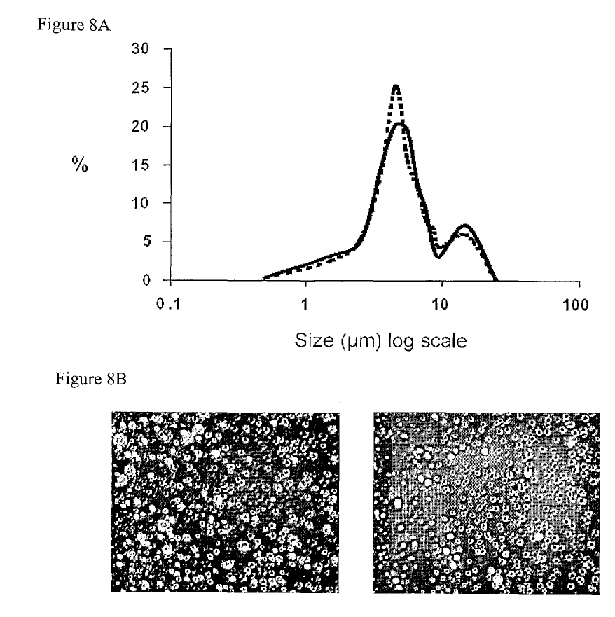

The present example indicates that optimal results, regarding stability of the emulsion during the PCR reaction, were obtained with an oil phase composed of 2% Abil® EM90 and 0.05% Triton X-100 in mineral oil. As shown in FIG. 8, the average size, determined by dynamic light scattering (panel A) and the appearance, determined by microscopy (panel B) of the emulsion remain unchanged after 32 cycles of PCR. Thus, no significant coalescence of water droplets occurred.

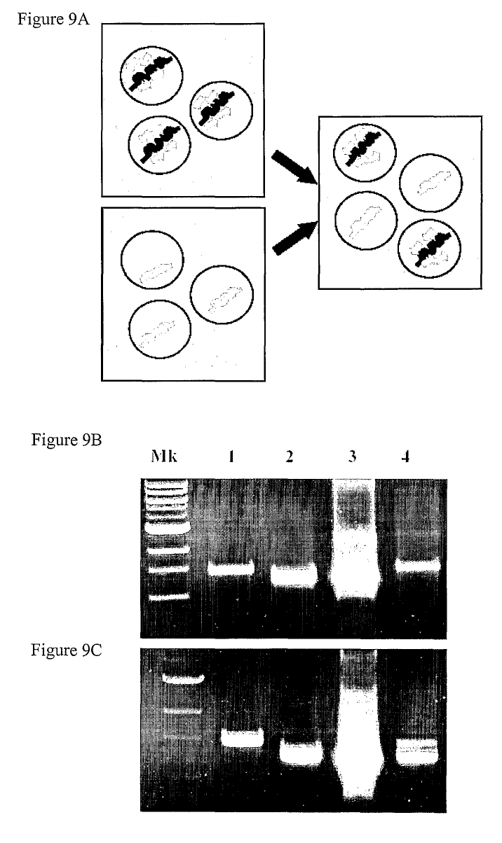

The stability of the Abil®-based emulsion was further analyzed in order to determine whether the water-phase components are properly sealed within individual droplets during the PCR cycles. For that, we prepared two separate emulsions, each one with a DNA template of different size, but both of which could be amplified with the same pair of primers (FIG. 9A). The emulsion containing the longer template (gray; primers shown as arrows) contained also all the components necessary for amplification, whereas the other was missing the primers (white). Prior to PCR, both emulsions were combined. Amplification product from an individual Abil® EM90-based emulsion containing the long DNA template is shown in FIG. 9B: lane 1; amplification product from an individual Abil EM90-based emulsion containing the short DNA template, lane 2; amplification product from the non-emulsified mixture of both templates, lane 3; amplification product of the separate Abil® EM90-based emulsions combined prior to PCR, lane 4. Control emulsions made of Span 80/Tween 80/Triton X-100 are shown in FIG. 9C. If droplets from the different emulsions mixed or water-based components shuttled within micelles between droplets, the short DNA template would come into contact with the primers and would be amplified. As shown in FIG. 9B, lane 3, only the long DNA template was amplified, indicating that the droplets were stable and no contents mixed. As controls, we confirmed that both DNA templates could be amplified in separate emulsions with the same pair of primers (FIG. 9C, lanes 1 and 2), and that when both templates are present together, either w/o emulsification (lane 3) or if mixed prior to emulsification (lane 4), the short DNA template is preferentially amplified.

On the contrary, when the same experiment was done on a regular 4.5% Span 80, 0.5% Tween 80, 0.05% Triton X-100, in mineral oil emulsion (in this case 100 μl of water phase are emulsified with 600 μl of oil phase), we observed that PCR cycling led to either water droplets coalescence or micellar exchange of water-based components, as indicated by the amplification of the short DNA template in FIG. 9, B, lane 4.

### Example 6

### Abil EM90-Based Emulsions Prevent Recombination Artifacts

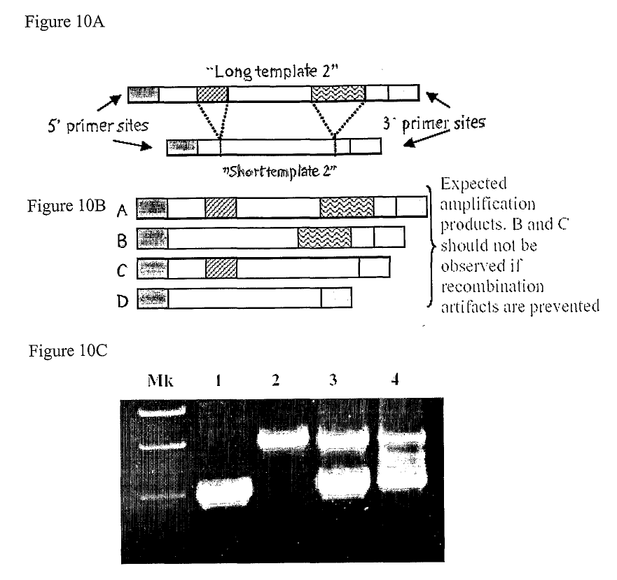

We determined whether single molecule PCR in Abil® EM90-based emulsions could be used to prevent recombination artifacts arising from premature termination during PCR elongation. For that, we built two DNA templates, one of which had two internal deletions, as indicated in FIG. 10A. The extensive common sequence ensures that recombination artifacts due to premature termination during extension could occur. The different sizes of the “chimeric” products facilitate their identification by agarose electrophoresis (FIG. 10B)

We prepared a single Abil EM90-based emulsion containing both DNA templates at a total concentration of ˜10⁸ molecules in 100 μl of water phase and subjected it to PCR cycling, as described above. As expected (FIG. 10B), no intermediate size bands, arising from recombination artifacts can be seen in lane 3 of FIG. 10C. On the contrary, when the same PCR mixture is amplified w/o emulsification (lane 4 of FIG. 10C) two bands of intermediate size are clearly visible. As controls, we tested that both templates could be amplified within emulsions (lanes 1 and 2).

Thus, we have developed a novel water-in-oil emulsion formulation ideal for PCR applications, including RT-PCR. Whilst this system is based on Abil EM90, other polymeric surfactants that are not affected by temperatures ≦94° C. might be applied in a similar way. Furthermore, the high stability of this emulsion system renders it highly suitable for the development of multiplex procedures for the isolation of single-cell DNA, RNA, or protein, as well as for single-cell based assays, at a population level.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A library of genetic elements encoding gene products, the library being compartmentalized in aqueous droplets of a water-in-oil emulsion, wherein each aqueous droplet comprises the components necessary to express gene products encoded by the genetic elements and further comprises at least one biological moiety the activity of which results in the modification of said genetic elements or the gene products encoded by said genetic elements.

2. The library of claim 1, wherein the at least one biologically active moiety is not activated.

3. The library of claim 1, wherein each aqueous droplet further comprises at least one activating agent capable of activating the biologically active moiety.

4. The library of claim 3, wherein the at least one biologically active moiety is selected from the group consisting of: a protein, a polypeptide and a peptide.

5. The library of claim 4, wherein the at least one biologically active moiety is an enzyme.

6. The library of claim 5, wherein the at least one biologically active moiety is a nuclease.

7. The library of claim 5, wherein the at least one activating agent is selected from the group consisting of: inorganic or organic salts, monosaccharides, disaccharides, oligosaccharides, amino acids, peptides, polypeptides, nucleotides, nucleosides, oligonucleotides, polynucleotides, vitamins and small organic molecules.

8. The library of claim 6, wherein the at least one activating agent is a bivalent salt.

9. A method for selecting genetic elements encoding gene products of a desired activity, the method comprising:
a) providing a library of genetic elements;
b) providing at least one biologically active moiety the activity of which results in the modification of said genetic elements or the gene products encoded by said genetic elements;
c) co-compartmentalizing the genetic elements with the at least one biologically active moiety into aqueous droplets, the aqueous droplets being the internal discontinuous phase of a water-in-oil emulsion, such that each aqueous droplet comprises at least one genetic element together with the at least one biologically active moiety and further comprises components necessary to express the gene products encoded by said at least one genetic element;
d) merging the water-in-oil emulsion with micelles comprising at least one activating agent capable of modulating the activity of said at least one biological moiety; and
e) detecting genetic elements encoding gene products having a desired activity.

10. The method of claim 9, further comprising prior to merging the water-in-oil emulsion with the micelles, the step of:
incubating the water-in-oil emulsion under conditions enabling expression of said gene products.

11. The method of claim 9, further comprising following merging the water-in-oil emulsion with the micelles, the steps of:
coalescing the water-in-oil emulsion thereby forming a continuous aqueous phase from the droplets; and
detecting in the aqueous phase genetic elements which encode the desired gene products.

12. The method of claim 9, wherein detecting the genetic elements comprises amplifying said genetic elements using PCR techniques and detecting the amplified products.

13. The method of claim 11, wherein the aqueous phase is re-emulsified prior to amplification.

14. The method of claim 11, wherein the aqueous phase is re-emulsified in oil comprising a surfactant capable of maintaining the integrity of the water-in-oil emulsion at temperatures within the range of 65° C. to 100° C.

15. The method of claim 14, wherein the surfactant is a polymer having a Hydrophilic-Lipophilic Balance value below 10.

16. The method of claim 14, wherein the surfactant is high molecular weight modified polyether polysiloxane.

17. The method of claim 14, wherein the surfactant is selected from the group consisting of: cetyl dimethicone copolyol, polysiloxane polyalkyl polyether copolymer, cetyl dimethicone copolyol, polyglycerol ester, poloxamer and polyvinyl pyrrolidone/hexadecane copolymer.

18. The method of claim 14, wherein the surfactant is cetyl dimethicone copolyol.

19. The method of claim 13, wherein the ratio of said surfactant to the oil is within the ranges of 1-20% v/v.

20. The method of claim 9, wherein the micelles comprise from 100 to 400 volumes of oil, and from 10 to 40 volumes of total surfactant to every one volume of an aqueous phase containing the at least one activating agent.

21. The method of claim 20, wherein the micelles have a mean droplet size in the range of 0.01 micron to 1 micron.

22. The method of claim 21, wherein the mean droplet size is approximately 0.1 micron.

23. The method of claim 9, wherein the at least one biologically active moiety is selected from the group consisting of: an enzyme, a protein, a polypeptide and a peptide.

24. The method of claim 23, wherein the biologically active moiety is a nuclease.

25. The method of claim 9, wherein the at least one activating agent is selected from the group consisting of: inorganic or organic salts, monosaccharides, disaccharides, oligosaccharides, amino acids, peptides, polypeptides, nucleotides, nucleosides, oligonucleotides, polynucleotides, vitamins and small organic molecules.

26. The method of claim 24, wherein the at least one activating agent is a bivalent salt.

27. A gene product selected according to the method of claim 9.

28. The gene product of claim 27, being a nuclease inhibitor.


SEQUENCE LISTING (4 sequences)

SEQ ID NO: 1 - DNA, 18 bp, artificial sequence
  PCR primer
  misc_feature (1)..(1): biotin at the 5′ position
caggctgcgcaactgttg

SEQ ID NO: 2 - DNA, 17 bp, artificial sequence
  PCR primer
gtcatagctgtttcctg

SEQ ID NO: 3 - DNA, 18 bp, Artificial sequence
  PCR primer
atgtgctgcaaggcgatt

SEQ ID NO: 4 - DNA, 17 bp, Artificial sequence
  PCR primer
gtcgatagtggctccaa